(19) United States
(12) Patent Application Publication
(10) Pub. No.: US 2022/0193331 A1
(43) Pub. Date: Jun. 23, 2022

(54) INFUSION BAG PRESSING DEVICE HAVING PRESSING BAG

(71) Applicant: Moung Sook LEE, Anyang-si (KR)

(72) Inventor: Moung Sook LEE, Anyang-si (KR)

(21) Appl. No.: 17/693,167

(22) Filed: Mar. 11, 2022

Related U.S. Application Data
(63) Continuation-in-part of application No. 16/487,476, filed on Aug. 21, 2019.

(30) Foreign Application Priority Data
Jan. 30, 2019 (KR) 10-2019-0012180

(51) Int. Cl. A61M 5/148

(52) U.S. Cl. CPC A61M 5/1486; A61M 5/168

(57) ABSTRACT

Provided is an infusion bag pressing device capable of pressing an infusion bag that accommodates an infusion solution, the infusion bag pressing device including: an air storage space configured to accommodate air; and a pressing bag configured to press the infusion bag by being expanded by the air flowing into the air storage space, in which the air storage space includes one or more unit space portions spatially separated by an interval maintaining member, and in which the interval maintaining member is disposed in the air storage space, one end of the interval maintaining member is coupled to an inner surface of one end of the pressing bag, and the other end of the interval maintaining member is coupled to an inner surface of the other end of the pressing bag, such that the pressing bag is inhibited from being excessively expanded.

### TECHNICAL FIELD

The present invention relates to an infusion bag pressing device, and more particularly, to an infusion bag pressing device capable of inhibiting a pressing bag from being excessively expanded or distorted.

### BACKGROUND ART

An infusion solution (fluid) is a liquid to be injected into a human body when shock, dehydration, malnutrition, or the like occurs. The infusion solution is a kind of medicine containing blood and various electrolyte solutions such as a physiological saline solution or a ringer solution having the same osmotic pressure as the blood.

The infusion solution (fluid) is a liquid to be injected into a patient's vein in case of a surgical operation, a treatment, shock, dehydration, malnutrition, or the like and contains blood, an electrolyte solution, and medicine similar to the blood. The infusion solution is a medicine containing a physiological saline solution, a ringer solution, a nutritional supplement, an intravenous injection solution, or the like having the same osmotic pressure as a human bodily fluid.

In the related art, an infusion bag (fluid bag), which is a medicine injection device for supplying the infusion solution, is hung upside down and positioned at a position higher than a human body. Further, the medicine is injected into the human body through an injection tube and an injection needle connected to the infusion bag.

A large amount of medicine needs to be injected emergently in an operating room or the like. However, the medicine injection device in the related art cannot be used for this purpose.

To solve this problem, a piston-type medicine injection device and a syringe-type medicine injection device have been developed. However, there is still a problem in that these medicine injection devices have complicated structures, are highly likely to be broken down, are difficult to use to inject a large amount of medicine, and incur a large amount of overall manufacturing cost.

The background art of the present application is disclosed in Korean Patent Application Laid-Open No. 10-2003-0095847.

### DISCLOSURE

### Technical Problem

The present invention has been made in an effort to solve the above-mentioned problems, and an object of the present invention is to provide an infusion bag pressing device having an improved structure capable of inhibiting a pressing bag from being excessively expanded or distorted.

### Technical Solution

To achieve the object, an infusion bag pressing device according to the present invention is capable of pressing an infusion bag that accommodates an infusion solution and includes: an air storage space configured to accommodate air; and a pressing bag configured to press the infusion bag by being expanded by the air flowing into the air storage space, in which the air storage space includes one or more unit space portions spatially separated by an interval maintaining member, and in which the interval maintaining member is disposed in the air storage space, one end of the interval maintaining member is coupled to an inner surface of one end of the pressing bag, and the other end of the interval maintaining member is coupled to an inner surface of the other end of the pressing bag, such that the pressing bag is inhibited from being excessively expanded.

In this case, the interval maintaining member may include a flexible member that cannot substantially resist a shearing force.

In this case, the interval maintaining member may include a band-shaped member extending in a longitudinal direction and having a predetermined thickness and a predetermined width.

The unit space portions may be configured to be expanded at a substantially equal expansion speed.

In this case, the interval maintaining member may extend along an imaginary first central axis extending from an upper end of the pressing bag to a lower end of the pressing bag, the interval maintaining member may be provided in plural, and the plurality of interval maintaining members may be arranged and spaced apart from one another at a predetermined interval along a second central axis that intersects the first central axis.

In this case, at least one of upper ends of the plurality of unit space portions and at least one of lower ends of the plurality of unit space portions may communicate with each other.

In this case, the infusion bag pressing device may further include an infusion bag accommodation portion configured to detachably accommodate the infusion bag.

In this case, the infusion bag may be integrally coupled to the pressing bag.

### Advantageous Effects

According to the present invention, an infusion bag pressing device, which is capable of pressing an infusion bag that accommodates an infusion solution, includes: an air storage space configured to accommodate air; and a pressing bag configured to press the infusion bag by being expanded by the air flowing into the air storage space, in which the air storage space includes one or more unit space portions spatially separated by an interval maintaining member, and in which the interval maintaining member is disposed in the air storage space, one end of the interval maintaining member is coupled to an inner surface of one end of the pressing bag, and the other end of the interval maintaining member is coupled to an inner surface of the other end of the pressing bag, such that the pressing bag is inhibited from being excessively expanded or distorted, and as a result, it is possible to innovatively reduce the pressing time, allow a user to easily and visually check the discharge amount of infusion solution without hindering a discharge flow of infusion solution unlike the related art, and completely discharge the infusion solution accommodated in the infusion bag.

According to the present invention, the pressing bag for pressing the infusion bag may be integrally coupled to the infusion bag, which makes it easy to manage the infusion solution (fluid) and makes it possible to improve convenience of use for a medical team and reduce overall manufacturing costs.

### DESCRIPTION OF DRAWINGS

FIG. 1 is a front view illustrating an infusion bag pressing device according to an embodiment of the present invention.

FIG. 2A is a rear view of the infusion bag pressing device illustrated in FIG. 1.

FIG. 2B is a rear view of the infusion bag pressing device having an interval maintaining member extending in a leftward/rightward direction.

FIG. 3 is a view for explaining a state in which the infusion bag pressing device illustrated in FIG. 1 is used.

FIG. 4 is a cross-sectional view taken along line A-A and illustrating the infusion bag pressing device illustrated in FIG. 1.

FIG. 5 is a cross-sectional view taken along line B-B in a state in which a pressing bag illustrated in FIG. 3 is expanded.

FIG. 6 is a cross-sectional view taken along lines C-C and D-D and illustrating the infusion bag pressing device illustrated in FIG. 1.

FIG. 7 is an enlarged perspective view of part VII of the infusion bag pressing device illustrated in FIG. 5.

FIGS. 8A and 8B are views illustrating an infusion bag pressing device according to another embodiment of the present invention.

### BEST MODE

Hereinafter, exemplary embodiments of the present invention will be described in detail with reference to the accompanying drawings.

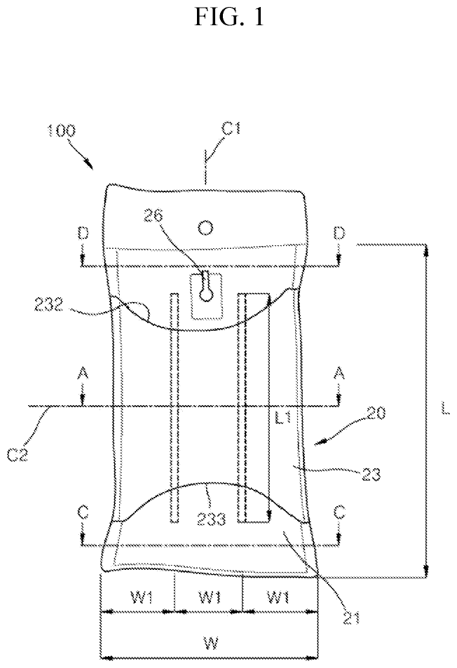

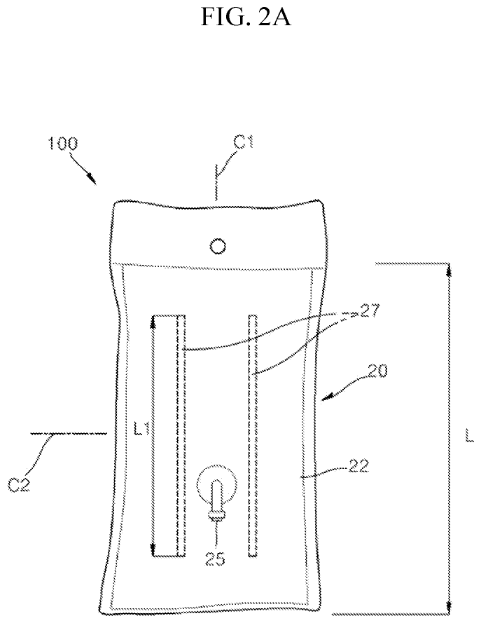

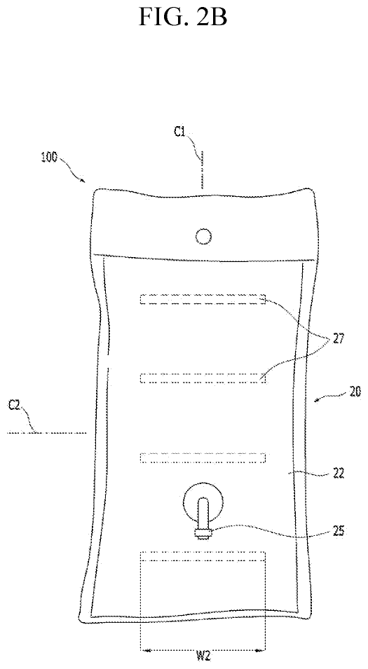

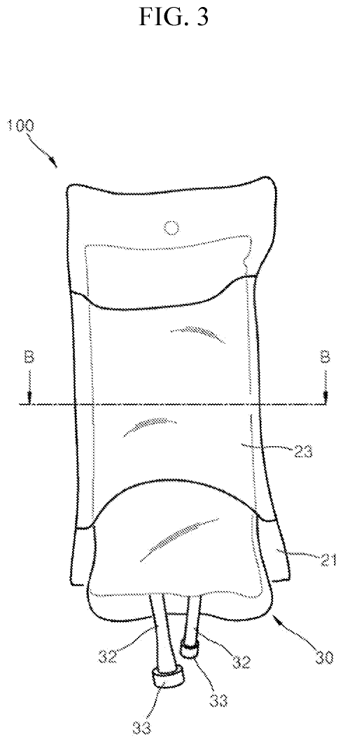

FIG. 1 is a front view illustrating an infusion bag pressing device according to an embodiment of the present invention, and FIG. 2 is a rear view of the infusion bag pressing device illustrated in FIG. 1. FIG. 3 is a view for explaining a state in which the infusion bag pressing device illustrated in FIG. 1 is used.

Referring to FIGS. 1 to 3, an infusion bag pressing device 100 according to an exemplary embodiment of the present invention is a device capable of discharging an infusion solution to the outside by pressing an infusion bag 30 that accommodates the infusion solution. The infusion bag pressing device 100 includes a pressing bag 20.

The infusion bag 30 will be described first before the pressing bag 20 is described.

The infusion bag 30 is a bag-type container for storing an infusion solution (medical fluid) to be injected into a human body and manufactured using a comparatively thin synthetic resin film.

In this case, the infusion solution (fluid) is a liquid to be injected into a patient's vein in case of a surgical operation, a treatment, shock, dehydration, malnutrition, or the like and contains blood, an electrolyte solution, and medicine similar to the blood. The infusion solution is a medicine containing a physiological saline solution, a ringer solution, a nutritional supplement, an intravenous injection solution, or the like having the same osmotic pressure as a human bodily fluid.

The infusion bag 30 may be manufactured using a flexible film made of a polystyrene-based material, a polyolefin-based material, a polyurethane-based material, a polyester-based material, vinyl chloride, or the like.

The infusion bag 30 includes an infusion solution storage space 31 capable of accommodating the infusion solution, infusion solution discharge ports 32 configured to discharge the infusion solution stored in the infusion solution storage space 31 to the outside, and cap parts 33 disposed at ends of the infusion solution discharge ports 32 and configured to seal the infusion solution discharge ports 32.

The infusion solution discharge port 32 is a tubular member disposed at a lower end of the infusion bag 30 and communicates with the infusion solution storage space 31.

The cap part 33 has a configuration into which a needle (not illustrated) connected to a drip chamber (not illustrated) may be inserted. Because the cap part 33 is widely known to those skilled in the art, a detailed description thereof will be omitted.

The infusion bag 30 may be a standard product widely used for medical purposes or a non-standard product.

According to the embodiment of the present application, an upper portion of the pressing bag 20 is manufactured using a comparatively thin synthetic resin film. A hanging hole for supporting the infusion bag pressing device 100 on a vertical structure or the like may be formed in a central portion of the upper portion of the pressing bag 20, but the present invention is not limited thereto.

The pressing bag 20 may be provided in the form of a bag that may press the infusion bag 30 by being expanded by air.

For example, the pressing bag 20 may be made of a material containing polyolefin-based resin, but the present invention is not limited thereto. In the description of the embodiment of the present application, the pressing bag 20 may be made of various materials each having hardness for supporting the infusion bag 30.

The pressing bag 20 includes a first sheet 21, a second sheet 22, a third sheet 23, an air storage space 24, an air injection port 25, an infusion bag hanger 26, and interval maintaining members 27. In this case, the pressing bag 20 has a predetermined horizontal length W and a predetermined vertical length L.

As illustrated in FIG. 1, the first sheet 21 may be disposed at a front side of the pressing bag 20. In addition, as illustrated in FIG. 2, the second sheet 22 may be disposed at a rear side of the pressing bag 20.

In addition, according to the embodiment of the present application, at least one of the first sheet 21 and the second sheet 22 may include a raw fabric made by fusing a film with a nylon material to improve a waterproof function and a windproof function, but the present invention is not limited thereto. As another example, at least one of the first sheet 21 and the second sheet 22 may include a raw fabric having similar properties to a material typically used for a cuff of a blood pressure meter or the like, but the present invention is not limited thereto.

The second sheet 22 may be a member disposed to face the first sheet 21 and have the same thickness and shape as the first sheet 21 in the present embodiment, but the present invention is not limited thereto. According to the embodiment of the present application, the first sheet 21 and the second sheet 22 may have different thicknesses, shapes, materials, and properties. In particular, in the case of an infusion bag pressing device 200 to be described below according to another embodiment of the present application in which the infusion bag 30 and the pressing bag 20 are integrally coupled, the first sheet 21 and the second sheet 22 may have different thicknesses in consideration of a load or the like of the infusion bag 30 integrally coupled to the pressing bag 20.

A rim of the first sheet 21 and a rim of the second sheet 22 are fused with each other by a fusing process, such that the air storage space 24 may be sealed. For example, the fusing process may include a thermal bonding method, but the present invention is not limited thereto. Various fusing methods of coupling the rim of the first sheet 21 and the rim of the second sheet 22 may of course be applied to the present application.

The third sheet 23 is disposed at a front side of the first sheet 21. For example, the third sheet 23 may be a semi-transparent or transparent synthetic resin film.

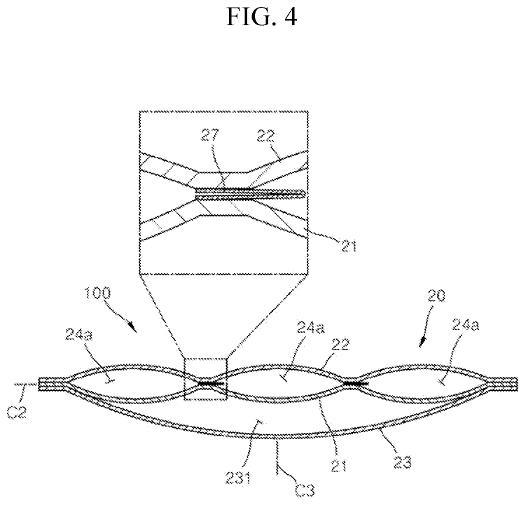

Two opposite left and right ends of the third sheet 23 are coupled to two opposite left and right ends of the pressing bag 20 by a fusing process, such that an infusion bag accommodation portion 231 is formed, as illustrated in FIG. 4.

The infusion bag accommodation portion 231 may be a space portion capable of detachably accommodating the infusion bag 30 therein. For reference, in the case of the infusion bag pressing device 200 to be described below in detail according to another embodiment of the present application, the infusion bag 30 and the pressing bag 20 are integrally coupled to each other, such that the infusion bag accommodation portion 231 may not be provided.

An upper end opening portion 232 is formed at an upper end of the infusion bag accommodation portion 231, and a lower end opening portion 233 is formed at a lower end of the infusion bag accommodation portion 231.

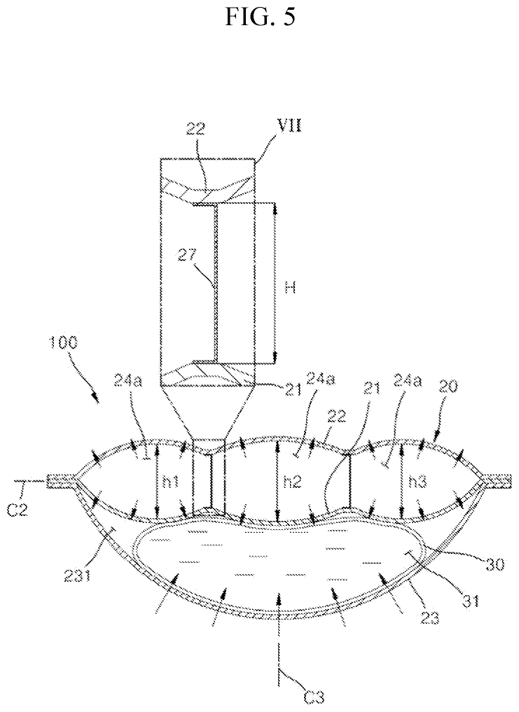

That is, the third sheet 23 is provided in the form of a belt coupled to two opposite left and right ends of the first sheet 21. As illustrated in FIG. 5, the third sheet 23 is a member capable of pressing the infusion bag 30.

Since the upper and lower ends of the infusion bag accommodation portion 231 are opened, a user may easily insert or withdraw the infusion bag 30 through the upper end opening portion 232 or the lower end opening portion 233 of the infusion bag accommodation portion 231.

The air storage space 24 is a space capable of accommodating air injected from the outside. In the present embodiment, an inner surface of the first sheet 21 and an inner surface of the second sheet 22 cooperatively define the air storage space 24.

The air injection port 25 is an air injection port communicating with the air storage space 24 and disposed at a center of a lower end of a rear surface of the pressing bag 20, as illustrated in FIG. 2.

The infusion bag hanger 26 is a looped member for hanging the infusion bag 30 and disposed at an upper end of a front surface of the pressing bag 20, as illustrated in FIG. 1.

The interval maintaining member 27 is a member configured to inhibit the pressing bag 20 from being excessively expanded during the process of expanding the pressing bag 20.

In the present embodiment, the interval maintaining member 27 is disposed in the air storage space 24. As illustrated in FIG. 5, one end of the interval maintaining member 27 is coupled to the inner surface of the first sheet 21 by a fusing process, and the other end of the interval maintaining member 27 is coupled to the inner surface of the second sheet 22 by a fusing process.

As illustrated in FIG. 4, the interval maintaining member 27 may be a flexible member that cannot substantially resist a shearing force.

However, the interval maintaining member 27 may sufficiently withstand a tensile force generated by an aerodynamic force.

In the present embodiment, the interval maintaining member 27 may be manufactured as a film made of the same synthetic resin material as the third sheet 23, but the present invention is not limited thereto.

Meanwhile, according to a first embodiment of the present application in respect to the arrangement of the interval maintaining members 27 of the infusion bag pressing device 100 or 200, the interval maintaining member 27 may rectilinearly extend in an upward/downward direction along an imaginary first central axis C1 extending from an upper end of the pressing bag 20 to a lower end of the pressing bag 20, as illustrated in FIGS. 1 and 2A. As another example, according to a second embodiment of the present application, the interval maintaining member 27 may rectilinearly extend in the leftward/rightward direction (the horizontal direction) along an imaginary second central axis C2 extending from a left end of the pressing bag 20 to a right end of the pressing bag 20, as illustrated in FIG. 2B.

Although not illustrated in the drawings, the plurality of interval maintaining members 27 according to the embodiments of the present application may be disposed at various intervals and various positions relative to the pressing bag 20, such that the interval maintaining members 27 are disposed in a diagonal direction (e.g., a direction from one o'clock to seven o'clock direction, a direction from eleven o'clock to five o'clock, or the like based on FIGS. 1A to 3) defined by the leftward/rightward direction and the upward/downward direction or the interval maintaining members 27 are disposed side by side or to cross one another (e.g., in an X (cross) shape or the like) in a predetermined direction inclined with respect to the leftward/rightward direction and the upward/downward direction.

Meanwhile, referring to FIGS. 1 and 2A, the plurality of interval maintaining members 27 is arranged and spaced apart from one another at a predetermined interval W1 along the second central axis C2 intersecting the first central axis C1, but the present invention is not limited thereto. According to the embodiments of the present application, different spacing intervals between the interval maintaining members 27 may be set for at least some of the pairs of interval maintaining members 27.

Likewise, referring to FIG. 2B, the interval maintaining members 27, which are disposed along the second central axis C2 (for example, referred to as a horizontal arrangement), may also be arranged and spaced apart from one another at a predetermined interval along the first central axis C1, but the present invention is not limited thereto. According to the embodiments of the present application, different spacing intervals between the interval maintaining members 27 may be set for at least some of the pairs of interval maintaining members 27.

In this regard, according to the embodiment of the present application, the interval maintaining members 27 rectilinearly extending along the second central axis C2 may be disposed such that the spacing interval between the interval maintaining members 27 gradually decreases downward so that the infusion solution accommodated in the infusion bag 30 is continuously discharged, and the infusion solution is easily pressed downward by the interval maintaining members 27 even though the amount of remaining infusion solution is relatively small during a process in which an upper portion of the pressing bag 20 (e.g., an upper region relative to the amount of infusion solution remaining in the infusion bag 30) is wound downward and presses downward the region of the infusion solution remaining in the infusion bag 30 to press the non-discharged infusion solution remaining a lower portion of the infusion bag 30. However, the present invention is not limited thereto.

In addition, according to the embodiment of the present application, some of the plurality of interval maintaining members 27 are configured as vertical members extending along the first central axis C1, and the remaining interval maintaining members 27 are configured as horizontal members extending along the second central axis C2, such that the plurality of interval maintaining members 27 may be disposed in a combination of the arrangement in the leftward/rightward direction (the horizontal arrangement) and the arrangement in the upward/downward direction (the vertical arrangement). For example, the vertical members may be disposed in the upper portion of the pressing bag 20, and the horizontal members may be disposed in the lower portion of the pressing bag 20, but the present invention is not limited thereto.

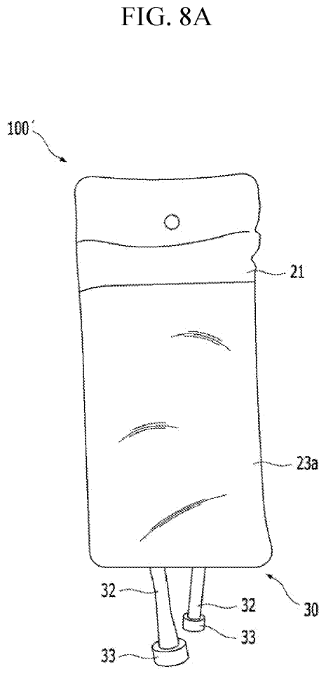

In the present embodiment, as illustrated in FIG. 7, the interval maintaining members 27 include a band-shaped member having a predetermined thickness T and a predetermined width H+2S.

In this case, the width H+2S of the interval maintaining member 27 determines a maximum spacing distance H between the first sheet 21 and the second sheet 22 when the pressing bag 20 is expanded maximally, as illustrated in FIG. 5.

Consequently, as illustrated in FIG. 5, the air storage space 24 includes one or more unit space portions 24a spatially separated by the interval maintaining members 27.

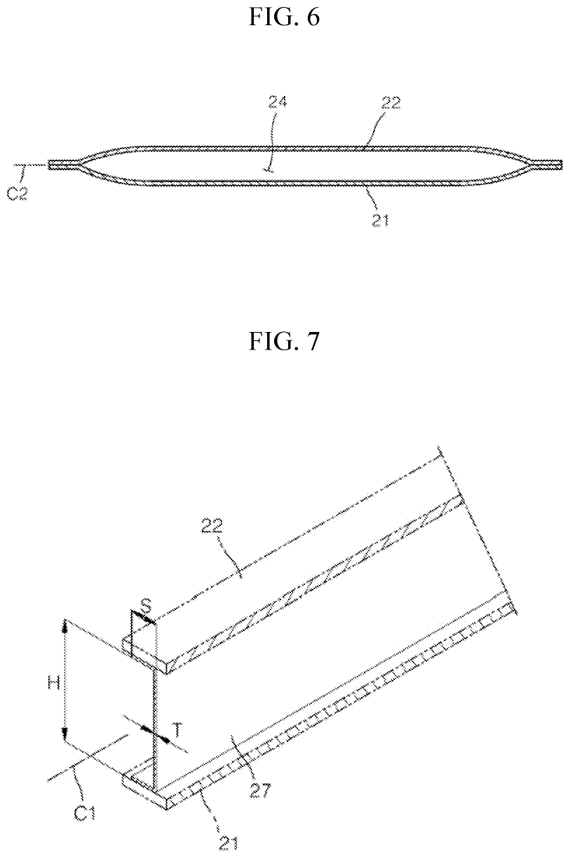

In the present embodiment, a length L1 of the interval maintaining member 27 is shorter than a length L of the pressing bag 20. Therefore, as illustrated in FIGS. 1 and 6, all the upper and lower ends of the plurality of unit space portions 24a communicate with one another.

In the present embodiment, all the upper and lower ends of the plurality of unit space portions 24a communicate with one another, all the unit space portions 24a having internal spaces with the same capacity, and the air injection port 25 is provided at the center of the lower end of the rear surface of the pressing bag 20. Therefore, the unit space portions 24a may be expanded at a substantially and almost equal expansion speed.

Hereinafter, an example of a method of using the infusion bag pressing device 100 or the infusion bag pressing device 200, which is configured as described above, will be described.

First, as illustrated in FIG. 3, the infusion bag 30 is mounted in the infusion bag accommodation portion 231, and the infusion bag 30 is fixed by being hung on the infusion bag hanger 26. However, since the infusion bag pressing device 200 to be described below according to another embodiment of the present application, in which the pressing bag 20 and the infusion bag 30 are integrally coupled, does not have the infusion bag accommodation portion 231, the process of mounting the infusion bag 30 in the infusion bag accommodation portion 231 may be omitted.

When the air is not injected into the air storage space 24 of the pressing bag 20, the pressing bag 20 is maintained in a non-expanded state, as illustrated in FIG. 4. In this case, the interval maintaining member 27 is also maintained in a folded state.

When a separate air injection device (not illustrated) is connected to the air injection port 25 and then the air injection device operates, the air is introduced into the air storage space 24, and the pressing bag 20 is expanded, as illustrated in FIG. 5.

The air injection device (not illustrated) may have various configurations such as a piston-type pump or an air pump operated by electricity, and a detailed description thereof will be omitted.

As the air is injected into the air storage space 24 of the pressing bag 20, the pressing bag 20 is gradually expanded, and the interval maintaining members 27 are tensely pulled, as illustrated in FIG. 5, and thus inhibit an interval H between the first sheet 21 and the second sheet 22 from increasing to a predetermined value or more.

In this case, the unit space portions 24a are expanded at a substantially and almost equal expansion speed because all the upper and lower ends of the plurality of unit space portions 24a communicate with one another, all the unit space portions 24a having internal spaces with the same capacity, and the air injection port 25 is provided at the center of the lower end of the rear surface of the pressing bag 20.

Further, unlike a pressing bag in the related art in which a middle portion of the pressing bag has a larger thickness than an edge portion of the pressing bag when the air is maximally injected into the air storage space 24, the thicknesses of the plurality of unit space portions 24a, which is separated by the interval maintaining members 27, may be maintained relatively uniformly, but the present invention is not limited thereto. According to the embodiments of the present application, the spacing intervals between the interval maintaining members 27 or the thicknesses of the interval maintaining members 27 are appropriately adjusted, such that a predetermined unit space portion 24a, among the plurality of unit space portions 24a, may be expanded to have a relatively larger or smaller thickness than the remaining unit space portion 24a.

When the air pressure in the air storage space 24 increases, a pressure for squeezing the pressing bag 20 is applied to the pressing bag 20 by the air pressure, as illustrated in FIG. 5. Therefore, the infusion solution stored in the infusion solution storage space 31 of the infusion bag 30 may be discharged to the outside through the infusion solution discharge port 32 by a difference between a pressure of the infusion solution stored in the infusion bag 30 and the outside atmospheric pressure.

The infusion solution discharged through the infusion solution discharge port 32 may be injected into a blood vessel via the cap part 33 and the drip chamber (not illustrated).

The infusion bag pressing device 100 configured as described above is a device capable of pressing the infusion bag 30 that accommodates the infusion solution, and the infusion bag pressing device 100 includes: the air storage space 24 configured to accommodate the air; and the pressing bag 20 configured to press the infusion bag 30 by being expanded by the air flowing into the air storage space 24. The air storage space 24 includes the one or more unit space portions 24a spatially separated by the interval maintaining members 27. The interval maintaining member 27 is disposed in the air storage space 24, one end of the interval maintaining member 27 is coupled to the inner surface of one end of the pressing bag 20, and the other end of the interval maintaining member 27 is coupled to the inner surface of the other end of the pressing bag 20. Therefore, the pressing bag 20 is inhibited from being excessively expanded or distorted in a forward/rearward direction C3, thereby reducing the pressing time.

As a result, since the use of the infusion bag pressing device 100 or 200 prevents the “distortion” of the pressing bag 20 and the infusion bag 30, the user may easily and visually check the discharge amount of the infusion solution without hindering the discharge flow of infusion solution unlike the related art. Unlike a case in which a large amount of air is required to allow a pressing pressure to reach a predetermined pressure level in a pressing bag of Patent No. 10-2070393 in the related art in which the interval maintaining member 27 is not provided and an air storage space is formed by directly bonding sheets, the infusion bag pressing device 100 or 200 disclosed in the present invention may advantageously allow a pressing pressure to easily reach a predetermined pressure level only by using a small amount of air and completely discharge the infusion solution accommodated in the infusion bag 30.

Further, according to the infusion bag pressing device 100, the interval maintaining member 27 includes the flexible member that cannot substantially resist the shearing force. Therefore, as illustrated in FIG. 4, in a state in which no air is injected into the pressing bag 20, the interval maintaining member 27 may be folded, which makes it possible to reduce an overall storage volume.

Meanwhile, in the case of the infusion bag pressing device 100 in which the unit space portions 24a are expanded at a substantially equal expansion speed, there is no risk that the pressing bag 20 is distorted while being expanded due to different expansion speeds. Further, the entire region of the infusion bag 30 may be pressed by a uniform pressure.

On the contrary, in the case of the infusion bag pressing device 100 in which some of the plurality of unit space portions 24a have different standards and expansion speeds from the remaining unit space portions 24a, the pressurization of the infusion solution accommodated in the infusion bag 30 may be further facilitated.

More specifically, in the case of the infusion bag pressing device 100 including the plurality of interval maintaining members 27 rectilinearly extending along the second central axis C2, the upper unit space portion, which is formed at an upper side among the plurality of unit space portions 24a formed by the interval maintaining member 27, is expanded preferentially, such that the unit space portion may press downward the infusion solution positioned at a relatively lower side by pushing and squeezing the infusion bag 30, thereby further reducing the pressing time.

In addition, as described above, according to the infusion bag pressing device 100, the plurality of interval maintaining members 27 extends along the imaginary first central axis C1 extending from the upper end of the pressing bag 20 to the lower end of the pressing bag 20 and is arranged (in other words, vertically arranged) and spaced apart from one another along the second central axis C2. Alternatively, the plurality of interval maintaining members 27 extends along the imaginary second central axis C2 extending from the left end of the pressing bag 20 to the right end of the pressing bag 20 and is arranged (in other words, horizontally arranged) and spaced apart from one another along the first central axis C1. As a result, the pressing bag 20 may be easily deformed to encircle and surround the infusion bag 30 while being expanded.

In addition, since the infusion bag pressing device 100 further includes the infusion bag accommodation portion 231 capable of detachably accommodating the infusion bag 30, the infusion bag pressing device 100 may be reused multiple times only by replacing the infusion bags 30.

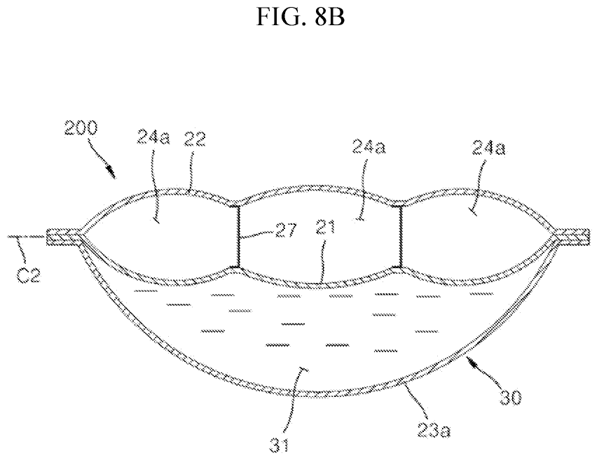

Meanwhile, FIGS. 8A and 8B illustrate the infusion bag pressing device 200 according to another embodiment of the present invention. Because the infusion bag pressing device 200 is identical mostly in configurations and effects to the infusion bag pressing device 100, only a difference between the two infusion bag pressing devices will be described below.

The infusion bag pressing device 200 does not include the infusion bag accommodation portion 231 which is a space portion capable of detachably accommodating the infusion bag 30 therein, but the infusion bag 30 is integrally coupled to the pressing bag 20.

The difference between the two infusion bag pressing devices is that the first sheet 21 and the third sheet 23a cooperatively define the infusion solution storage space 31 of the infusion bag 30.

Therefore, unlike the infusion bag pressing device 100 which may be reused, the infusion bag pressing device 200 is a single-use device. However, because a medical team need not perform a separate process of mounting the infusion bag, the medical team may quickly treat an emergent patient, and there is no risk that a medical accident such as cross-contamination occurs.

In the present embodiment, the interval maintaining member 27 includes a flexible film-shaped member that cannot substantially resist a shearing force. Alternatively, the interval maintaining member 27 may of course be a member in the form of a beam that may resist shearing and bending forces with predetermined magnitudes. When the interval maintaining member 27 is a member in the form of a beam, the pressing bag 20 may appropriately withstand an external bending force applied to the center of the second central axis C2.

In the present embodiment, as illustrated in FIG. 1, the interval maintaining member 27 rectilinearly extends along the imaginary first central axis C1 extending from the upper end of the pressing bag 20 to the lower end of the pressing bag 20. Alternatively, the interval maintaining member 27 may of course rectilinearly extend along the second central axis C2 that intersects the first central axis C1.

Meanwhile, according to the embodiment of the present application, in the case of the pressing bag 20 in which the interval maintaining member 27 rectilinearly extends along the second central axis C2, the infusion solution accommodated in the infusion bag 30 is continuously discharged, and the upper portion of the pressing bag 20 (e.g., the upper region relative to the amount of infusion solution remaining in the infusion bag 30) is wound downward and presses downward the region of the infusion solution remaining in the infusion bag 30 corresponding to the non-discharged infusion solution remaining at the lower side of the infusion bag 30 as the amount of remaining infusion solution decreases, thereby facilitating the discharge of the remaining infusion solution. In this regard, the upper portion of the pressing bag 20 may be wound downward by being wound in a rounded shape in the upward/downward direction about the interval maintaining member 27, for example.

In the present embodiment, the unit space portions 24a are arranged from the left end of the pressing bag 20 to the right end of the pressing bag 20. Alternatively, the unit space portions 24a may of course be arranged from the upper end of the pressing bag 20 to the lower end of the pressing bag 20.

In the present embodiment, the interval maintaining member 27 is made of a film material that does not transmit air, like the first sheet 21. Alternatively, the interval maintaining member 27 may of course be a member made of a material that transmits air.

In the present embodiment, the pressing bag 20 is manufactured using a polyethylene film. Alternatively, the pressing bag 20 may of course be manufactured using other materials.

In the present embodiment, various elements of the infusion bag pressing device 100 are fused with one another by thermal bonding processes. Alternatively, various fusion or bonding processes such as high-frequency fusion and bonding fusion may of course be used.

In the present embodiment, the pressing bag 20 is formed by superimposing and bonding the two films including the first sheet 21 and the second sheet 22. Alternatively, the pressing bag 20 may of course be formed using a single pipe-shaped film. In this case, it is possible to reduce the number of times the thermal bonding process is performed.

While the present invention has been described, it is apparent that the technical scope of the present invention is not limited to the above-mentioned embodiments and equivalent configurations modified or changed by those skilled in the art do not depart from the technical spirit of the present invention.

1. An infusion bag pressing device capable of pressing an infusion bag that accommodates an infusion solution, the infusion bag pressing device comprising:
an air storage space configured to accommodate air; and
a pressing bag configured to press the infusion bag by being expanded by the air flowing into the air storage space,
wherein the air storage space comprises one or more unit space portions spatially separated by an interval maintaining member, and
wherein the interval maintaining member is disposed in the air storage space, one end of the interval maintaining member is coupled to an inner surface of one end of the pressing bag, and the other end of the interval maintaining member is coupled to an inner surface of the other end of the pressing bag, such that the pressing bag is inhibited from being excessively expanded.

2. The infusion bag pressing device of claim 1, wherein the interval maintaining member comprises a flexible member that cannot substantially resist a shearing force.

3. The infusion bag pressing device of claim 1, wherein the interval maintaining member comprises a band-shaped member extending in a longitudinal direction and having a predetermined thickness and a predetermined width.

4. The infusion bag pressing device of claim 1, wherein the unit space portions are configured to be expanded at a substantially equal expansion speed.

5. The infusion bag pressing device of claim 1, wherein the interval maintaining member extends along an imaginary first central axis extending from an upper end of the pressing bag to a lower end of the pressing bag, the interval maintaining member is provided in plural, and the plurality of interval maintaining members is arranged and spaced apart from one another at a predetermined interval along a second central axis that intersects the first central axis.

6. The infusion bag pressing device of claim 5, wherein at least one of upper ends of the plurality of unit space portions and at least one of lower ends of the plurality of unit space portions communicate with each other.

7. The infusion bag pressing device of claim 1, further comprising:
an infusion bag accommodation portion configured to detachably accommodate the infusion bag.

8. The infusion bag pressing device of claim 1, wherein the infusion bag is integrally coupled to the pressing bag.
